(19) United States
(12) Patent Application Publication
(10) Pub. No.: US 2024/0008753 A1
(43) Pub. Date: Jan. 11, 2024

(54) METHODS AND SYSTEMS FOR MULTI-PARAMETER HEMODYNAMIC MONITORING

(71) Applicants: UNIVERSITY OF PITTSBURGH - OF THE COMMONWEALTH SYSTEM OF HIGHER EDUCATION, Pittsburgh, PA (US); UNIVERSITY OF MARYLAND, College Park, MD (US)

(72) Inventors: Ramakrishna MUKKAMALA, Sewickley, PA (US); Sanjeev Govinddas SHROFF, Pittsburgh, PA (US); Michael R. PINSKY, Pittsburgh, PA (US); Aman MAHAJAN, Pittsburgh, PA (US); Kathirvel SUBRAMANIAM, Sewickley, PA (US); Vishaal DHAMOTHARAN, Pittsburgh, PA (US); Jin-Oh HAHN, Potomac, MD (US)

(73) Assignees: UNIVERSITY OF PITTSBURGH - OF THE COMMONWEALTH SYSTEM OF HIGHER EDUCATION, Pittsburgh, PA (US); UNIVERSITY OF MARYLAND, College Park, MD (US)

(21) Appl. No.: 18/219,185

(22) Filed: Jul. 7, 2023

Related U.S. Application Data
(63) Continuation of application No. PCT/US2022/011697, filed on Jan. 7, 2022.
(60) Provisional application No. 63/162,387, filed on Mar. 17, 2021.
(60) Provisional application No. 63/135,425, filed on Jan. 8, 2021.

(51) Int. Cl. A61B 5/021; A61B 5/022; A61B 5/00

(52) U.S. Cl. CPC A61B 5/02141; A61B 5/02225; A61B 5/7267; A61B 5/02108; G16H 10/60

(57) ABSTRACT

Devices and methods for multi-parameter hemodynamic monitoring are provided. Determining a cardiac output of a patient using a cuff device includes measuring a cuff pressure waveform of a subject during inflation and/or deflation of the cuff device, computing systolic and diastolic blood pressure from the cuff pressure waveform using the cuff device, constructing a blood pressure waveform from the systolic and diastolic blood pressure and cuff pressure waveform using the cuff device, computing brachial artery compliance from the cuff pressure waveform, and computing the cardiac output from the blood pressure waveform and/or brachial artery compliance using the cuff device.

### CROSS-REFERENCE TO RELATED APPLICATION

This application is a continuation of International Patent Application No. PCT/US2022/011697, filed Jan. 7, 2022, which claims priority to U.S. Provisional Patent Application Ser. No. 63/135,425, filed Jan. 8, 2021, and U.S. Provisional Patent Application Ser. No. 63/162,387, filed Mar. 17, 2021, all of which are hereby incorporated by reference herein in their entireties.

### BACKGROUND

Hypotension leading to circulatory shock can be an issue in surgical and intensive care. For example, during surgery, low blood pressure (BP) can occur at least in part due to the loss of blood and can be a harbinger of mortality in the initial post-operative month. Intensive care patients can develop septic shock, for example due to hypotension induced by infection in the bloodstream, and can be associated with increased mortality.

Monitoring BP can be useful for managing hypotension. But additional parameters, such as cardiac output (CO) and left ventricular ejection fraction (EF), can be beneficial, including to diagnose the etiology of hypotension to select the proper therapy. CO and EF can also help titrate therapy. For example and without limitation, in patients undergoing major surgery or with COVID-19-induced acute respiratory distress syndrome, fluids can be given in selected amounts according to these parameters to avoid or prevent deleterious over-resuscitation. If a fluid bolus causes CO to increase, then the patient can benefit from more fluid. If the fluid bolus causes no CO change, then the patient can be harmed by additional fluid. CO-directed fluid therapy can reduce mortality, morbidity, and length of hospital stay.

Certain devices can be used for hemodynamic monitoring of surgery and intensive care patients but can lack non-invasive and automated measurement of BP, CO, and EF with convenient instrumentation. For example, some pulse contour and volume-clamp devices can be based on BP waveform measurement. Yet while such devices can be suitable for high-risk patients with a clinical indication for arterial catheterization, which is less than 50% of all patients, such devices do not measure EF. Volume-clamp devices can measure a finger BP waveform and likewise estimate CO from this waveform. Such devices can be non-invasive, but can involve using or adapting a special, servo-controlled finger cuff embedded with an optical sensor. Moreover, such devices can be ineffective during episodes of finger hypo-perfusion.

Certain pulmonary artery catheter, ultrasound, and impedance cardiography devices do not monitor BP and are based on different measurement principles. Pulmonary artery catheter devices can measure CO and right ventricular ejection fraction using thermodilution methods as well as pulmonary pressures. However, such devices can be considered highly invasive. Doppler ultrasound devices measure CO, and echocardiography devices can measure CO and EF while imaging the heart. But these and other devices can involve a trained operator to steadily direct a trans-thoracic probe or frequently reposition a trans-esophageal probe. Impedance cardiography devices can measure CO non-invasively but with special, current-injecting electrodes on the neck and thorax. Such devices can be inaccurate due at least in part to interference from lung fluids.

By contrast, automatic arm cuff devices can be employed for non-invasive BP monitoring in surgery and intensive care patients including those with arterial catheters in place. However, arm cuff devices can lack multi-parameter hemodynamic monitoring and can be inaccurate at estimating BP from the cuff pressure waveform due at least in part to the use of population average algorithms. Although certain automatic wrist cuff devices, which can permit ambulatory monitoring, can be employed for intensive care patients, such devices can present similar challenges as arm cuff devices, as well as potential error due to hydrostatic effects and motion artifact.

Therefore, there is an opportunity for methods and systems for non-invasive and automated co-measurement of BP, CO, and EF with convenient instrumentation.

### SUMMARY

The purpose and advantages of the disclosed subject matter will be set forth in and are apparent from the description that follows, as well as will be learned by practice of the disclosed subject matter. Additional advantages of the disclosed subject matter will be realized and attained by the devices particularly pointed out in the written description and claims hereof, as well as from the appended drawings.

To achieve these and other advantages and in accordance with the purpose of the disclosed subject matter, as embodied and broadly described, the disclosed subject matter provides devices and methods for multi-parameter hemodynamic monitoring. A method for determining a cardiac output of a patient using a cuff device can include measuring a cuff pressure waveform of a subject during inflation and/or deflation of the cuff device, computing systolic and diastolic blood pressure from the cuff pressure waveform using the cuff device, constructing a blood pressure waveform from the systolic and diastolic blood pressure and cuff pressure waveform using the cuff device, computing brachial artery compliance from the cuff pressure waveform, and computing the cardiac output from the blood pressure waveform and/or brachial artery compliance using the cuff device.

In certain embodiments, the cuff pressure waveform can be measured during slow cuff inflation and/or deflation of the cuff device, followed by maintaining a cuff pressure at a sub-diastolic level. In non-limiting embodiments, a rate of the slow cuff inflation and/or deflation can be from about 2 to about 3 mmHg/second, and additionally or alternatively, the cuff pressure at the sub-diastolic level can be from about 40 to about 60 mmHg for about 10 to about 30 seconds.

In certain embodiments, the cardiac output can be computed based on additional information of the subject. In non-limiting embodiments, the additional information can include age, height, weight, gender of the subject, or combinations thereof.

In certain embodiments, the brachial artery compliance can be determined based on a ratio of peak-to-peak amplitude of the sub-diastolic pulse volume plethysmography (PVP) waveform to a pulse pressure or a ratio of peak-to-peak amplitude of the PVP waveform at a fixed transmural pressure to the pulse pressure.

In certain embodiments, the additional information, blood pressure values, and/or formula to compute arterial compliance factor from the brachial artery compliance can be defined through training data.

In certain embodiments, a common feature value can be estimated from the constructed blood pressure waveform, and wherein the cardiac output can be computed based on the common feature value.

In certain embodiments, the disclosed subject matter provides a method for determining a cardiac output of a subject using a cuff device. The method can include measuring the cuff pressure waveform during cuff inflation and deflation of the cuff device, computing an ensemble-averaged beat of a blood pressure waveform from the cuff pressure waveform, and computing the cardiac output from the ensemble-averaged blood pressure waveform beat.

In certain embodiments, the method can further include computing brachial artery compliance from the cuff pressure waveform using the cuff device. In non-limiting embodiments, the cardiac output can be computed based on additional information of the subject. The additional information can include artery compliance, age, height, weight, gender of the subject, or combinations thereof.

In certain embodiments, the ensemble-averaged beat can be formed from beats in a low cuff pressure range or from beats around or near a zero transmural pressure range.

In certain embodiments, the brachial artery compliance can be determined based on a ratio of peak-to-peak amplitude of a pulse volume plethysmography (PVP) waveform at a fixed transmural pressure to pulse pressure.

In certain embodiments, a formula to compute the cardiac output from the brachial artery compliance, blood pressure values, blood pressure waveform beat features, and/or the additional information can be defined through training data.

In certain embodiments, the blood pressure waveform is constructed without maintaining a cuff pressure at a sub-diastolic level.

In certain embodiments, the disclosed subject matter provides a method for determining the left ventricular ejection fraction of a subject using a cuff device. The method can include measuring a cuff pressure waveform during cuff inflation and deflation of the cuff device, computing a systolic and diastolic blood pressure from the cuff pressure waveform using the cuff device, constructing a blood pressure waveform from the systolic and diastolic blood pressure and the cuff pressure waveform, and computing the left ventricular ejection fraction from systolic and diastolic intervals of the constructed blood pressure waveform.

In non-limiting embodiments, the method can further include imaging the left ventricular ejection fraction for computing a subsequent left ventricular ejection fraction in the subject. In some embodiments, the subject can have a dilated heart.

In certain embodiments, the left ventricular ejection fraction can be determined from the constructed blood pressure waveform using classical or deep machine learning.

In certain embodiments, the left ventricular ejection fraction can be determined from a constructed blood pressure waveform using a physiologic model representing a left ventricle and arterial system.

In certain embodiments, the left ventricle system can be characterized by a variable elastance model.

In certain embodiments, the disclosed subject matter provides a method for determining the pulse pressure variation of a subject using a cuff device. The method can include measuring a cuff pressure waveform of the subject while maintaining a cuff pressure at a sub-diastolic level, detecting a peak-to-peak amplitude of each beat of the cuff pressure waveform, computing a respiratory period of the cuff pressure waveform, and computing the pulse pressure variation from the peak-to-peak amplitudes and respiratory period.

In certain embodiments, the disclosed subject matter provides a method for determining a systolic and diastolic blood pressure using a cuff device. The method can include measuring a cuff pressure waveform of a subject during at least one of inflation and deflation of the cuff device, constructing an oscillogram as a function relating variable cuff pressure oscillation amplitudes to cuff pressure, representing the oscillogram with a parametric model with parameters comprising the systolic and diastolic blood pressure and defining a brachial artery compliance curve, fitting the parametric model to the oscillogram to estimate the model parameters, and determining the systolic and diastolic blood pressure and the brachial artery compliance curve using the model parameter values. The brachial artery compliance curve is further represented with an exponential-linear parametric function,

### BRIEF DESCRIPTION OF THE DRAWINGS

FIG. 1 is a diagram illustrating an example arm cuff device in accordance with the disclosed subject matter.

FIG. 2 is a diagram illustrating example physiologic modeling methods of estimating multiple parameters using an example arm cuff device in accordance with the disclosed subject matter.

FIGS. 3A-3B are diagrams illustrating example patient-specific techniques for BP estimation in accordance with the disclosed subject matter.

FIGS. 4A-4E are diagrams illustrating example physiologic techniques for measurement of central BP via an example arm cuff device in accordance with the disclosed subject matter.

FIGS. 5A-5B are diagrams illustrating example pulse contour and multi-beat analysis techniques for CO estimation from a BP waveform in accordance with the disclosed subject matter.

FIGS. 6A-6D are diagrams illustrating example model-based techniques for EF estimation from a BP waveform in accordance with the disclosed subject matter.

FIG. 7 is a diagram illustrating example patient data for a training dataset in accordance with the disclosed subject matter.

FIG. 8 is a diagram illustrating example cuff pressure pre-processing for use with an arm cuff device in accordance with the disclosed subject matter.

FIG. 9 is a diagram illustrating example machine learning and physiologic techniques for estimating BP, CO, and EF from a cuff pressure waveform in accordance with the disclosed subject matter.

FIG. 10 is a graph illustrating an example plot of an expo-linear model in accordance with the disclosed subject matter.

### DETAILED DESCRIPTION

Reference will now be made in detail to the various exemplary embodiments of the disclosed subject matter, which are illustrated in the accompanying drawings.

The disclosed subject matter provides techniques for multi-parameter hemodynamic monitoring. The disclosed subject matter provides systems and methods for simultaneous monitoring of blood pressure (BP), cardiac output (CO), and left ventricular ejection fraction (EF) using a non-invasive cuff device. As referred to herein, such a cuff device can be applied to the arm. Additionally or alternatively, such a cuff device can be applied to other parts of a body, including but not limited to, a wrist, a finger, a leg, or any other suitable location for hemodynamic monitoring.

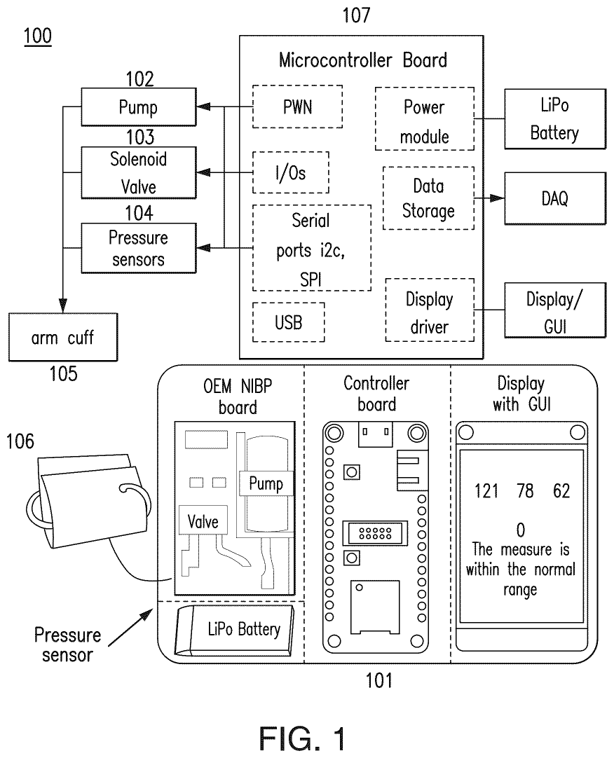

In certain embodiments, the disclosed subject matter provides arm cuff devices for multi-parameter hemodynamic monitoring. The arm cuff devices can estimate or measure one or more parameters, including but not limited to BP, CO, and EF, in a single wearable device. An example arm cuff device 100, as shown in FIG. 1, can have a board 101 that can further include an air pump 102, a valve 103, a pressure sensor 104, an inflatable cuff 105, tubing 106, a processor, and a microcontroller 107. For example, the arm cuff device can be built based on a board that can include an air micropump, a solenoid pressure valve, and a micromanometer. The pump can input air into the cuff via the tubing, the valve can allow the release of air from the cuff, and the pressure sensor can permit precise control of air transfer while providing a cuff pressure waveform to estimate the measured parameters.

As embodied herein, for purpose of illustration and not limitation, all components can be housed within a 3D-printed encasing.

In certain embodiments, the board can be programmed to control the cuff pressure. For example, the disclosed cuff devices can vary the external pressure of the brachial artery via cuff inflation/deflation and measure the pressure inside the cuff. The cuff pressure can equal the external pressure plus oscillations, indicating the pulsatile blood volume in the artery. The amplitude of the oscillations can vary with the cuff pressure, as the arterial blood volume-transmural pressure relationship can be nonlinear. In non-limiting embodiments, a transmural pressure can be defined as the internal BP minus the external cuff pressure. For purpose of illustration only, and as embodied herein, the board can interface with the microcontroller and integrate with a data storage unit, display driver, batteries. Alternatively, one or more of these devices can be provided as external components in communication with the microcontroller. The microcontroller can be portable and can include a touch screen. The microcontroller can be configured to quickly inflate the cuff to a supra-systolic pressure (e.g., not above a pre-set value), deflate to a sub-diastolic cuff pressure at a predetermined rate (e.g., about 2-3 mmHg/sec), and maintain a sub-diastolic cuff pressure (e.g., about 40-60 (±1) mmHg for about 10-30 sec). The microcontroller can utilize the preceding supra-systolic level when available and confirm supra-systolic and sub-diastolic levels by identifying a lack of a peak in the heart rate regime of the cuff pressure waveform spectrum. For purpose of illustration and not limitation, the microcontroller can be programmed to repeat the inflation/deflation cycle at user-specified intervals or at pre-programmed intervals. For example, a graphical user interface (GUI) can be designed for the touch screen to allow the user to enter the desired intervals and initiate or terminate a cycle on demand while displaying the instantaneous cuff pressure in real-time. The touch screen can display the hemodynamic variables.

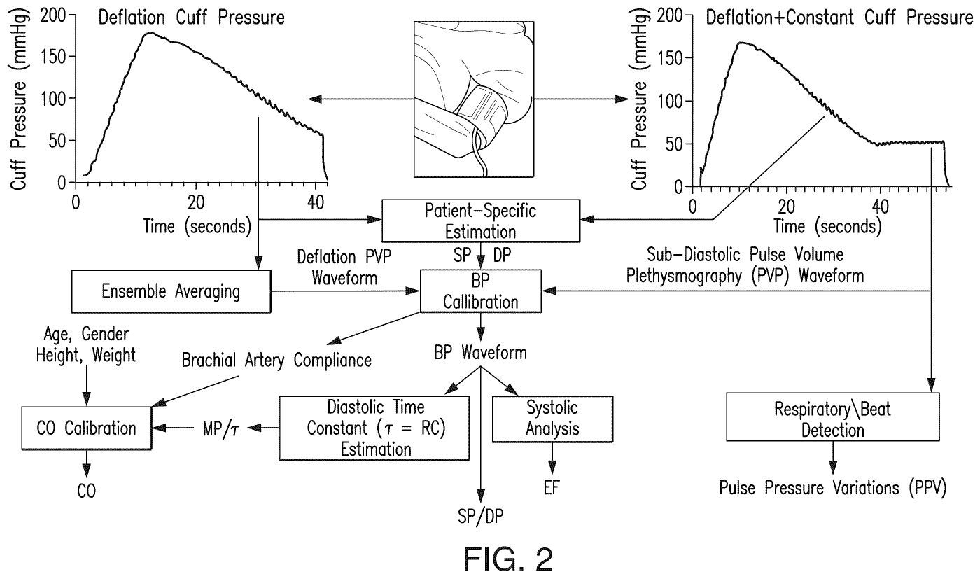

For example and as embodied herein, the board can include a processor, which can be configured to estimate BP, CO, and EF using a cuff pressure waveform. The processor can be configured to estimate a BP waveform using systolic/diastolic BP (SP/DP) from the cuff pressure waveform measured during the deflation period (e.g., in an oscillogram) and estimating CO, EF, and other hemodynamic variables using the BP waveform. For example and without limitation, as shown in FIG. 2, SP/DP can be estimated from the oscillogram formed from the cuff pressure waveform measured during the deflation period. A brachial BP waveform can be extracted from the same waveform or by subsequently measuring a more accurate but less convenient waveform via a sub-diastolic cuff pressure and calibrating the pulse volume plethysmography (PVP) waveform to SP/DP. CO and EF can be estimated from the BP waveform and basic patient information. As embodied herein, the cuff device can also measure changes in arterial compliance (C) for the CO computation, as the cuff pressure waveform can include both blood volume and BP information. For example and without limitation, brachial artery compliance can be estimated via a patient-specific technique or as the ratio of the sub-diastolic PVP amplitude to pulse pressure (PP=SP−DP) as described herein. In this manner, the cuff device can have improved accuracy over other devices, such as pulse contour/volume-clamp devices, which track C changes via BP alone. Additionally or alternatively, the cuff device can detect pulse amplitude variations from the sub-diastolic PVP waveform. Such pulse pressure variations (PPV) can predict fluid responsiveness in patients undergoing non-protective mechanical ventilation and in sinus rhythm. As such, whether a subject would benefit from fluid therapy can be determined without giving a fluid bolus to the subject.

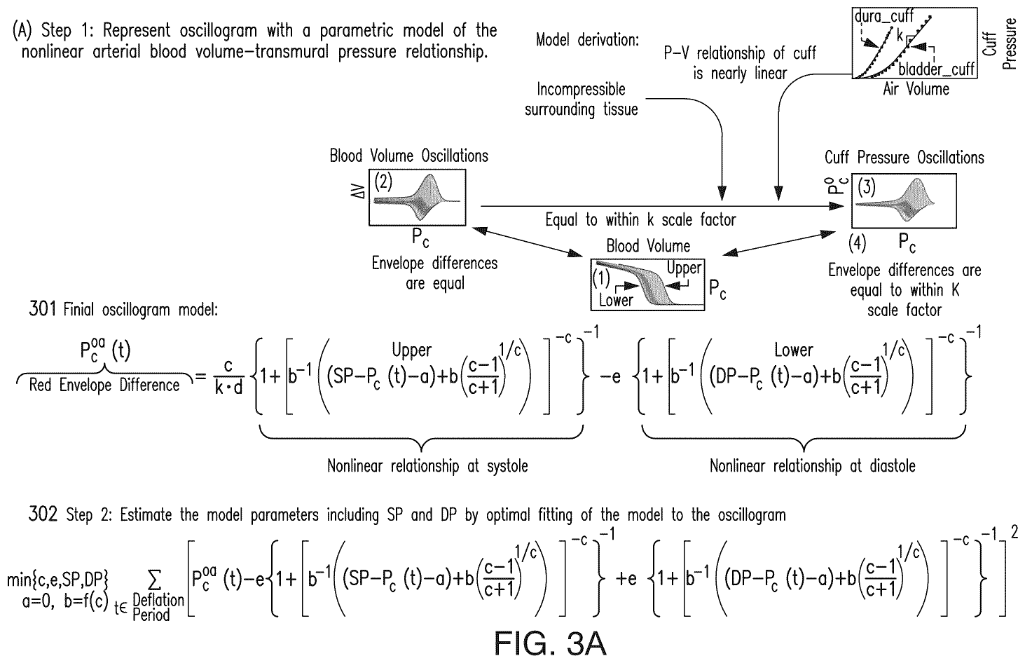

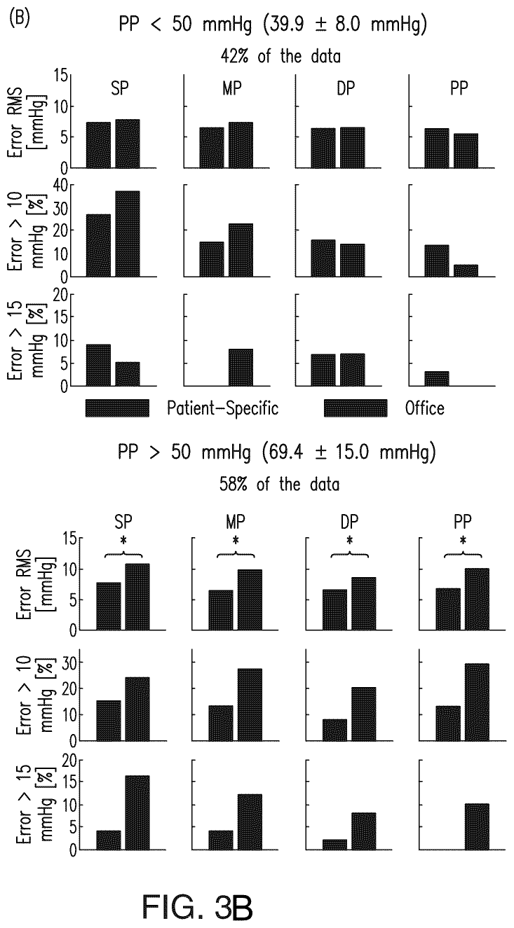

For purpose of illustration and not limitation, as embodied herein, the processor can perform various techniques for estimating BP, CO, and EF using a cuff pressure waveform. For example, the processor can be configured to estimate BP from the oscillogram obtained using the disclosed cuff device. The technique for estimating BP can be a patient-specific technique. The patient-specific technique can represent the oscillogram with a physiologic model and estimate the model parameters, which can represent SP and DP and define arterial stiffness, by fitting the model to the oscillogram. For example, as shown in FIG. 3A, the oscillogram, indicated by the vertical difference between the two red envelopes, can be represented with a model accounting for the nonlinear arterial blood volume-transmural pressure relationship, with reference to the Final oscillogram model 301 in FIG. 3A. Referring still to FIG. 3A, using the oscillogram model, patient-specific parameters, which represent SP and DP and define arterial stiffness (e.g., a, b, c, e in the Final oscillogram model), can be estimated by least-squares fitting of the model to the oscillogram 302. In non-limiting embodiments, certain parameters can be defined to improve the estimation. For example, parameter a can be equal or near 0 mmHg, parameter b can be defined based on parameter c such that the derivative of the nonlinear blood volume-transmural pressure relationship with respect to pressure (i.e., compliance curve) can be right-skewed by about 30-40%. Additionally or alternatively, other estimation methods known in the art and other parametric compliance curve models can be employed (e.g., parametric compliance curve models).

As embodied herein, various parametric functions can be used for representing a compliance curve. For example and without limitation, and as embodied herein, a Weibull function or an exponential-linear function can be used to represent a branchial artery compliance curve.

In certain embodiments, the step of computing a systolic and diastolic blood pressure or an ensemble-averaged beat of a blood pressure waveform from the cuff pressure waveform using the cuff device can involve representing the brachial artery compliance curve with an exponential-linear function or Weibull function. The so-parameterized brachial artery compliance curve (i.e., compliance as a function of transmural pressure) can be computed with BP from the cuff pressure waveform to improve accuracy.

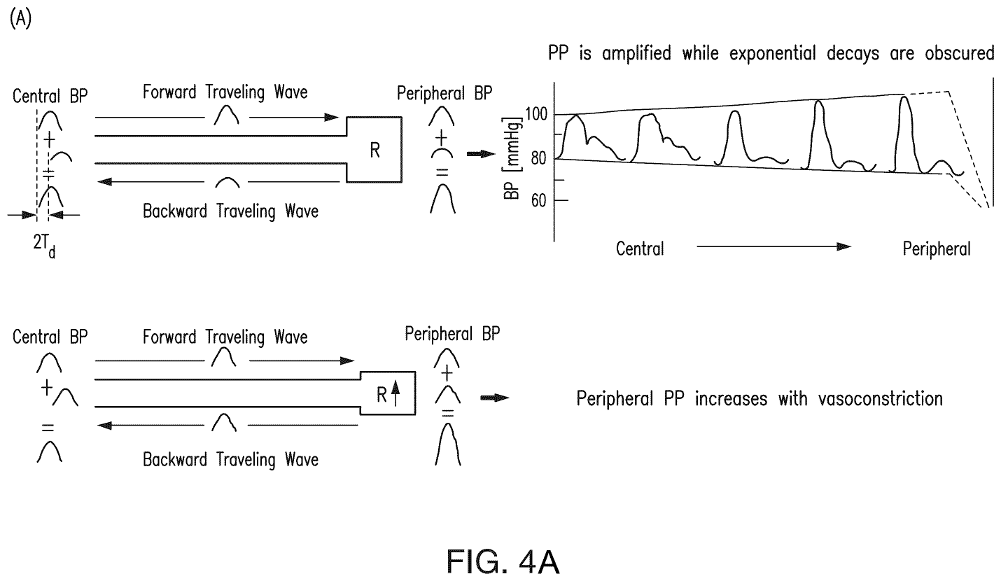

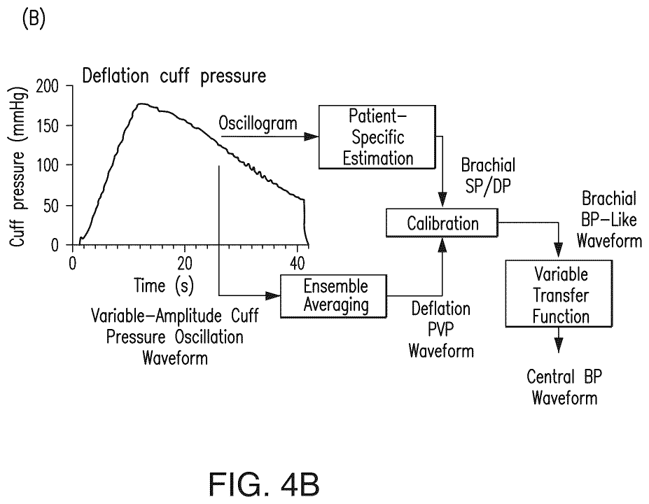

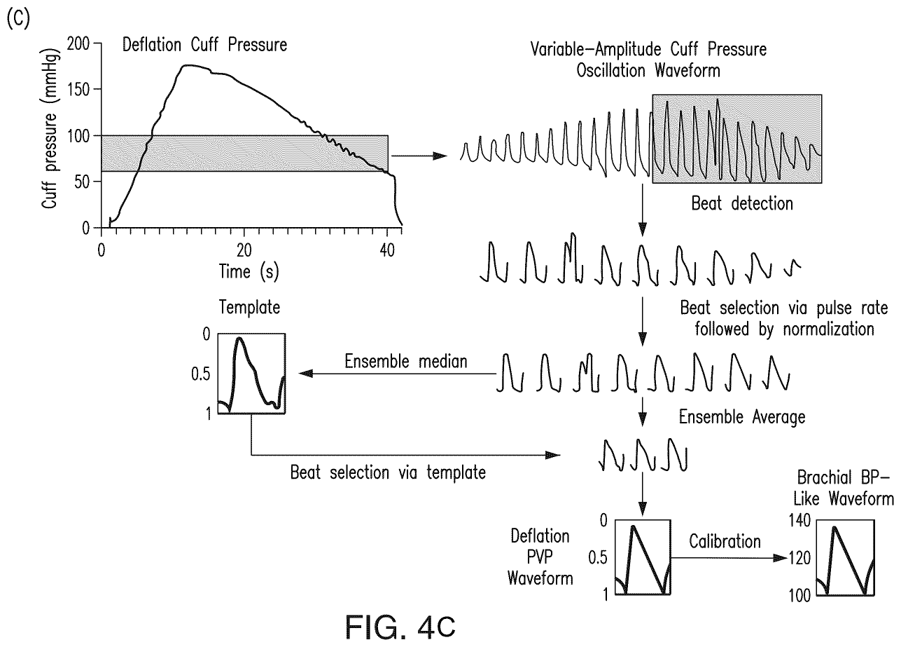

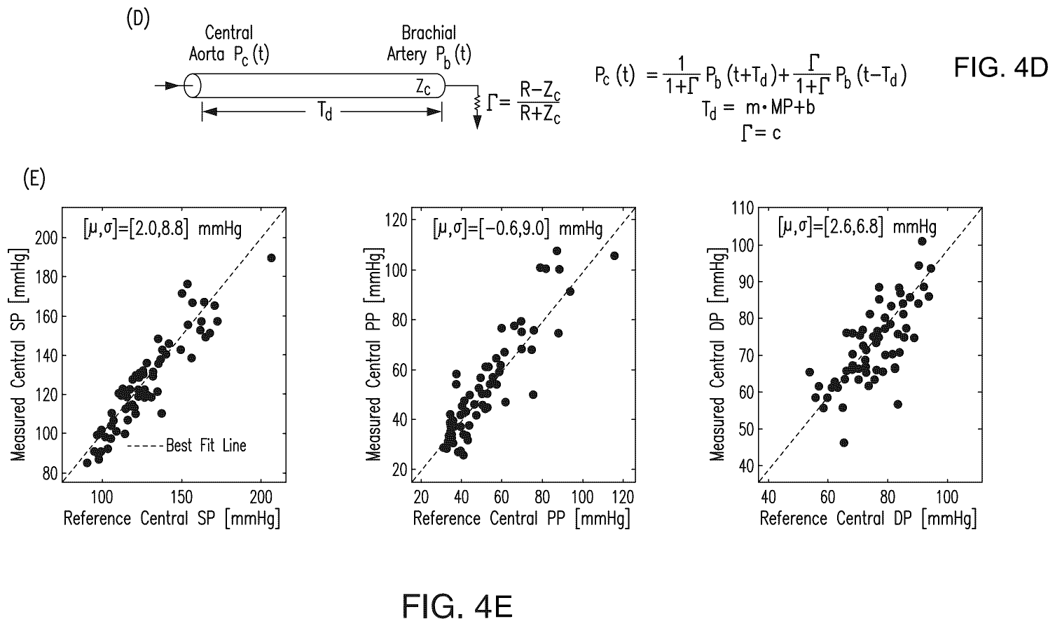

Further, for example and as embodied herein, the processor can perform techniques for measuring central BP. The technique for measuring central BP can measure central BP indirectly from the cuff pressure waveform. The technique for measuring central BP can be a physiologic technique. The physiologic technique can involve constructing a brachial BP waveform from the cuff pressure waveform during the deflation period and converting this waveform to the central BP waveform via a variable transfer function model of wave reflection. As shown for example in FIG. 4, the patient-specific technique can be applied to estimate brachial SP and DP, and an ensemble averaging/calibration technique can be applied to derive a brachial BP-like waveform. For example, a constant-amplitude cuff pressure waveform or a PVP waveform can be extracted from the variable-amplitude cuff pressure waveform, which can be performed by ensemble averaging of normalized waveform beats in amplitude and/or time over the lower cuff pressure regime or the transmural pressure range around 0 mmHg (e.g., about 0-30 mmHg, wherein the artery can be most linear due to dominance of elastic fibers in this range). The transmural pressure can be determined with the BP estimate (e.g., MP) of the patient-specific or other techniques by subtracting the cuff pressure from the BP. The ensemble averaging can exclude anomalous beats using a median beat and based on the pulse rate. In this manner, as embodied herein, a BP waveform for cardiac output estimation can be constructed without maintaining the cuff pressure at a sub-diastolic level.

Referring still to FIGS. 4A-E, for illustration and not limitation, the brachial BP-like waveform represented as Pb(t) can be converted into the central BP waveform represented as Pc(t) using a variable transfer function. The transfer function can be defined using a tube-load model of wave reflection with parameters representing pulse transit time [Td] and the wave reflection coefficient [Γ], which can represent transfer function in time-domain as shown for example in FIG. 4D. In some embodiments, the F parameter can be a nominal value, as the transfer function can be insensitive to it, while Td, which can affect the extent to which the transfer function reduces BP amplification, can be predicted based on its inverse relationship with mean BP (MP). The MP can be used to predict Td, and the defined transfer function can be applied to this waveform to derive the central BP waveform represented by Pc(t). In non-limiting embodiments, other available information including pulse rate, SP, DP, basic patient information such as age height, weight, gender, and cardiovascular risk factors, or combinations thereof can be used for more accurate prediction of Td and F.

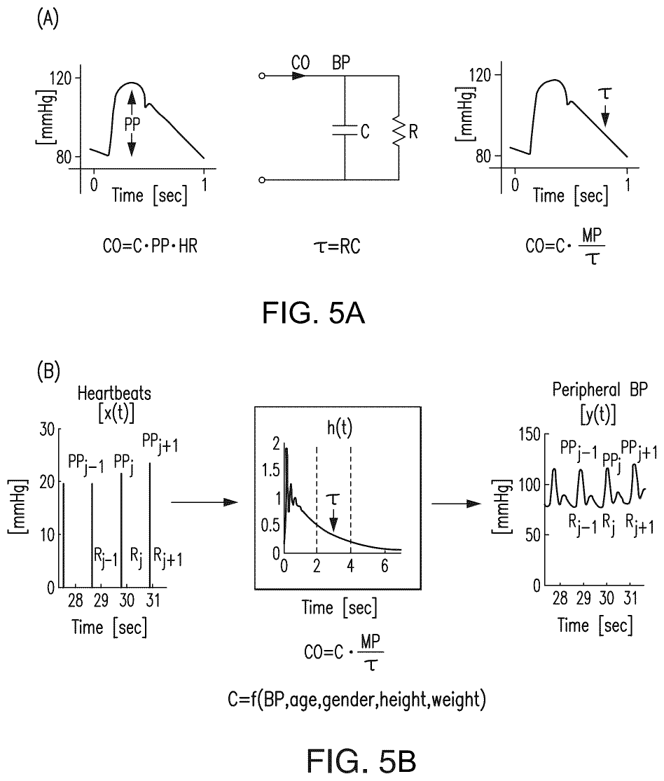

As embodied herein, the processor can include a multi-beat analysis technique for CO estimation from a BP waveform obtained with the cuff device, as shown for example in FIG. 5. The multi-beat analysis technique can model confounding wave reflection, which can cause pulse pressure (PP=SP−DP) to change irrespective of CO due to vasoconstriction/vasodilation leading to decreased accuracy. For example, as shown in FIG. 5B, the BP response to a single heartbeat can be estimated from a waveform segment over multiple beats. For illustration and not limitation, the single heartbeat BP response can be estimated. As embodied herein, a waveform indicating the heartbeats x(t) can be formed as an impulse train such that each impulse can be located at the minimum or foot of the BP waveform y(t) and scaled by ensuing high pulse pressure (PP=SP−DP). An impulse response h(t) can be identified, which when convolved with x(t) can best fit y(t) via autoregressive exogenous input least-squares identification or another similar technique. The impulse response h(t) represents the BP response to a single heartbeat. A resistor and capacitor (RC) time constant (t) can be determined by fitting an exponential to the tail end of this response when the faster wave reflection vanishes.

An arterial compliance factor (C) can be determined from BP, patient age, height, weight, and gender using a formula. CO can be determined by Ohm's law, as shown for example in FIGS. 5A-5B.

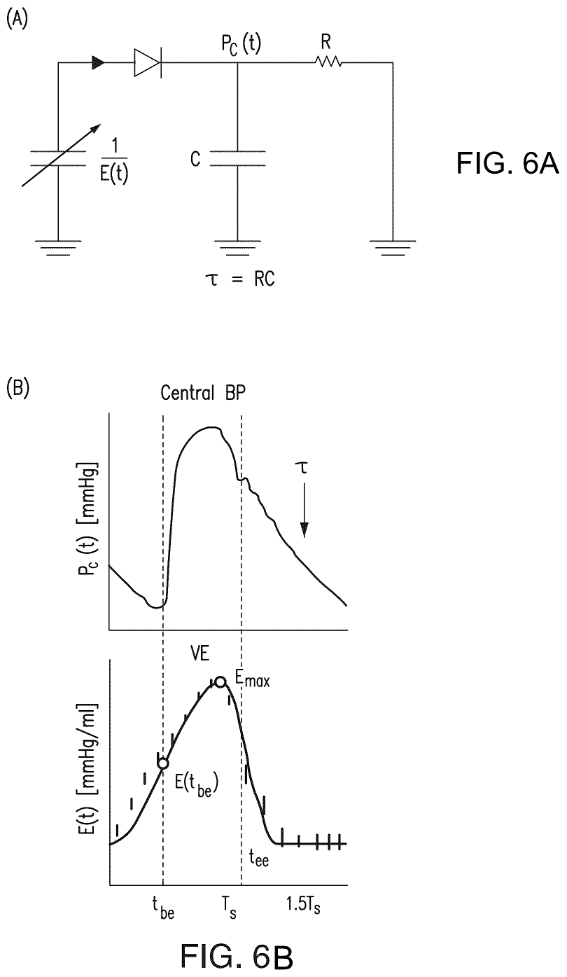

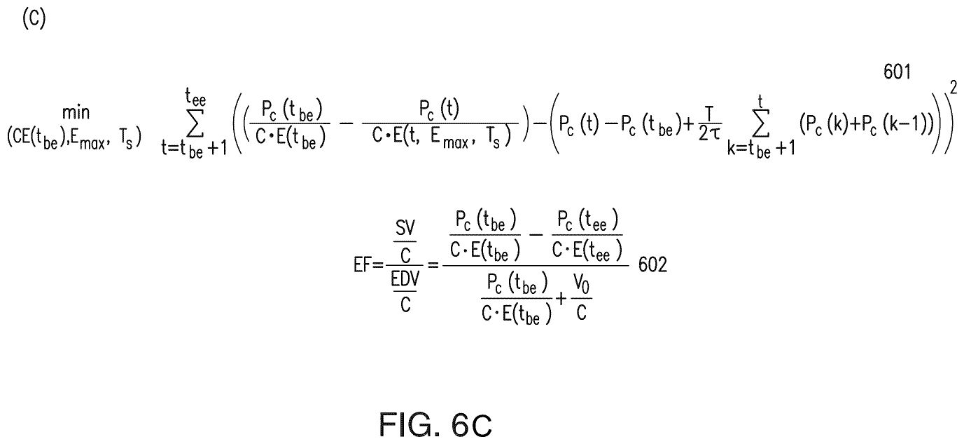

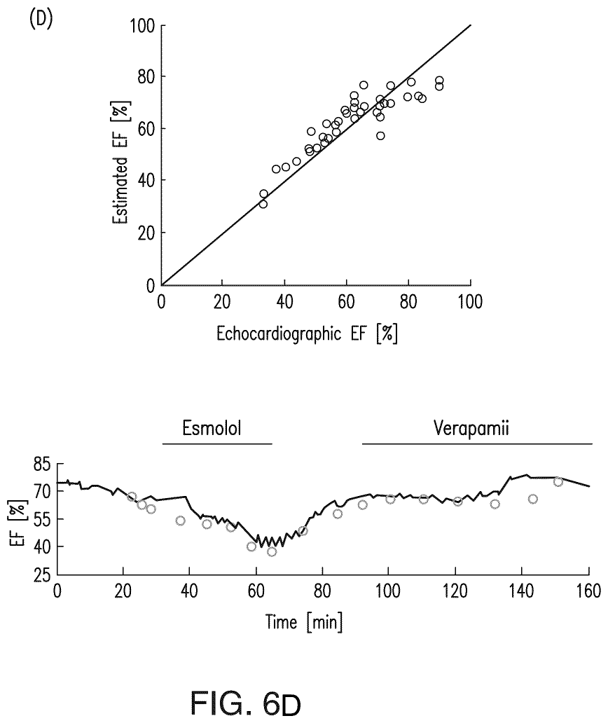

Further, and as embodied herein, the processor can include a model-based technique for EF estimation from a BP waveform obtained with the cuff device. For example, the model-based technique can model both the left ventricle and arteries. The model can be fit to the entire BP waveform to estimate the model parameters to within C scale factors. For purpose of illustration and not limitation, as shown in FIGS. 6A-6C, the model-based technique can be configured for application to the derived central BP waveform, which can exhibit exponential diastolic decays. The BP waveform Pc(t) can be represented with a lumped model of the left ventricle and arteries, as shown for example in FIG. 6A. The ventricle can include a variable elastance (VE) model, which can be a reciprocal of compliance, and in which elastance can vary over time E(t) to drive blood flow. The aortic valve can be modeled with a diode, and the arteries can be represented by an RC circuit model (e.g., a Windkessel model). As shown for example in FIG. 6B, E(t) can be parameterized using a raised cosine. The model can include parameters such as τ, CEₘₐₓ, Ts, and CE(t_{be}), where Eₘₐₓ is the maximum VE over a cardiac cycle, Ts is the time duration to reach Eₘₐₓ from the minimum VE, and the is the start time of the ejection interval, as shown for example in FIGS. 6A-6C. These parameters can be estimated from Pc(t). For example, can be determined by least-squares fitting of an exponential to its diastolic interval, as shown in FIG. 6B. CEₘₐₓ, Tₛ, and CE(t_{be}) can be estimated from the ejection interval of Pc(t) and ti by least squares matching of both sides of the governing model equation, as shown for example at 601 in FIG. 6C. EF can be determined from the CE(t) estimate and Pc(t) by deriving stroke volume (SV) and ventricular end-diastolic volume (EDV) to within C scale factors based on the model equations and taking the ratio of SV to EDV to nullify the common C term for example as shown at 602 in FIG. 6C. As embodied herein, the model-based technique can continuously monitor EF via an arterial waveform. The C term can be canceled out in the ensuing computation of EF to yield an absolute measurement in units of percent. A imaging measurement of EF can be used to determine the V₀/C term at 602 in FIG. 6C for patients with dilated hearts and can be assumed to take on a small constant value in other patients. The imaging can be performed periodically (e.g., every week) to improve accuracy.

In certain embodiments, the disclosed methods for determining the blood pressure, cardiac output, left ventricular ejection fraction, and/or pulse pressure variation of a subject using the cuff device can be based on physiologic modeling. In non-limiting embodiments, an oscillogram model including a nonlinear blood volume-transmural pressure relationship, an RC circuit model of the arteries, a single contraction blood pressure response model, a wave reflection model relating a peripheral blood pressure to a central blood pressure, and/or a variable elastance model of the ventricle can be used. The parameters of the model(s) can be determined by fitting the model to the cuff pressure measurement and/or using a training dataset as described herein. The blood pressure, cardiac output, left ventricular ejection fraction, and/or pulse pressure variation of a subject can then be computed in part from the parameters.

As embodied herein, combinations of the disclosed techniques can be used for estimating various hemodynamic variables. For example and without limitation, any of the techniques described herein can be combined in a cuff device for multi-parameter hemodynamic monitoring according to the disclosed subject matter.

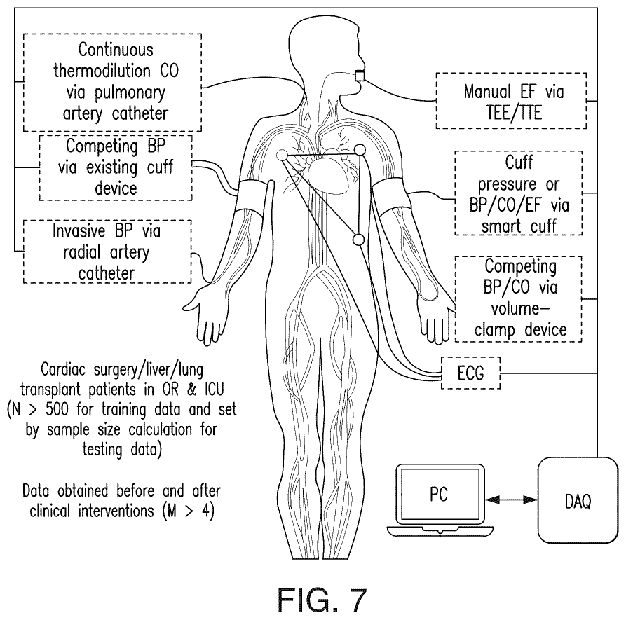

According to other aspects of the disclosed subject matter, the processor can be trained, for example using training data. In some embodiments, the processor can receive and evaluate training data. Additionally or alternatively, the training data can be received and evaluated by another device, such as an external computing device. The processor or external device can evaluate the received training data to develop a formula or model therefrom, and if performed by the external device, the processor can then receive the developed formula or model, which can used to estimate the hemodynamic variables described herein. The training data can be obtained using the cuff device or any other device or combination of devices suitable to measure the parameters described herein, for example as shown in FIG. 7. Such other devices can include, for purpose of illustration only and not limitation, a radial artery catheter for invasive BP, a pulmonary artery catheter for continuous/bolus thermodilution CO, a TEE probe for manual EF, electrodes for ECG, and a mechanical ventilator. As embodied herein, the inclusion criteria of the training data can be data of adults with radial/brachial and pulmonary artery catheters and a trans-esophageal echocardiography (TEE) probe in place as part of clinical care. Further, as embodied herein, the exclusion criteria of the training data can be significant aortic regurgitation, mechanical cardiac support, or significant tricuspid regurgitation. In non-limiting embodiments, the training data can be used to optimize methods for estimating BP, CO, EF, and pulse pressure variations as follows.

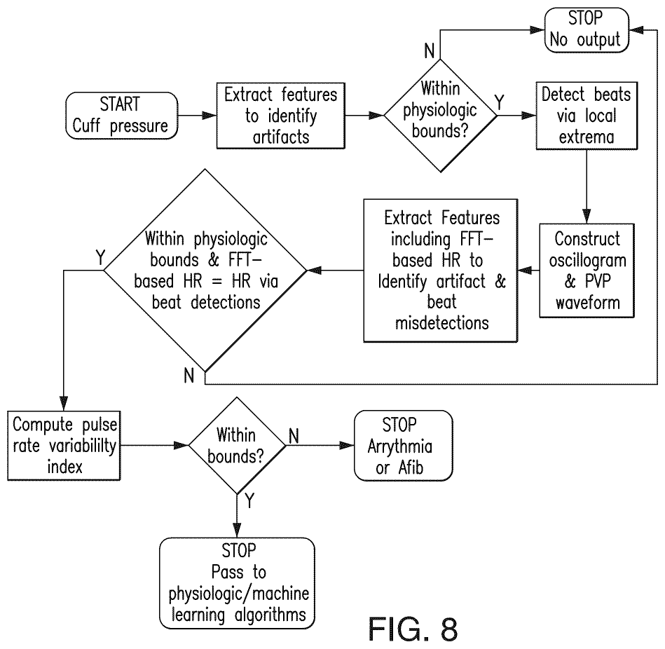

For purpose of illustration only and not limitation, the processor can screen the cuff pressure waveform for artifact and arrhythmia, for example as shown in FIG. 8. For example, the cuff pressure waveform can be screened for gross artifact based on expected cuff pressure waveform features. If an artifact is not found, each waveform beat can be detected via local extrema plus physiologic rules. Missed artifact or poorly detected beats can be identified via a non-physiologic oscillogram, such as oscillograms lacking an inverted U shape or lacking correspondence between the beats of the sub-diastolic PVP waveform and the peak location of its spectrum. The presence of atrial fibrillation (AF) or another major arrhythmia can be determined based on the pulse rate variability. If an arrhythmia or artifact is not found, techniques can be applied to estimate the hemodynamic parameters.

If arrhythmia is detected, the processor can output that an arrhythmia can be found instead of BP or other hemodynamic variables. Alternatively, for example and without limitation, a regression model can be used to predict each pulse amplitude of the sub-diastolic PVP waveform using the two preceding pulse intervals and using the model to subtract AF-induced variability in the oscillogram, and then the hemodynamic variables can be estimated. If an artifact is found, the processor can give no output.

For purpose of illustration and not limitation, the patient-specific technique can be configured to generate output values within a suitable time period for estimating BP, such as in less than 2 seconds following cuff deflation. Heuristic methods (e.g., fixed ratios) can be used to determine the cuff pressure range used for model fitting, which can improve the speed of the device. A nonlinear model of the cuff pressure-air volume relation defined by parameters (e.g., measured beforehand from the cuff) can be used to refine accuracy.

Furthermore, and as embodied herein, the multi-beat analysis technique and the physiologic technique can be optimized for estimating CO. For example, using the training data, the multi-beat analysis technique can be tailored to the BP-calibrated sub-diastolic PVP waveform, and the physiologic technique can be optimized to fit an exponential to a derived central BP diastolic decay.

For purpose of illustration and not limitation, a pulse contour technique can be provided as described herein. For example, changes in C can be tracked using the compliance estimate(s) of the patient-specific technique or using the ratio of the sub-diastolic PVP amplitude to PP or using the ratio of the PVP amplitude during the deflation or inflation period but at a fixed cuff pressure (e.g., 100 or 30 mmHg) or fixed transmural pressure (e.g., 0 or 70 mmHg) to PP. The computed ratios can be averaged over several beats near the fixed cuff or transmural pressure to mitigate noise. The absolute value of C and CO can be determined by creating a nomogram to predict CO from patient data. For example, the training data, which can include, without limitation, cuff device and reference CO data from patients during changes in CO and BP, can be used to define a regression or another equation to predict C from the compliance estimate, MP or other BP values, or patient, age, height, weight, and gender. Then, the so-obtained C can be multiplied by MP/r, where r is determined by the multi-beat analysis technique or otherwise, to determine CO via Ohm's law. As another example, the training data can be used to define a regression or another equation including a neural net to predict CO from the compliance estimate, MP or other BP values, τ or common features in the BP waveform such as area-under-the-curve, and patient, age, height, weight, gender. As a third example, the training data can be used to define an equation to predict CO from the entire BP waveform, the compliance estimate, and patient, age, height, weight, gender, or combinations thereof.

For estimating EF, the model-based technique can be adapted to the central BP waveform derived from the physiologic technique. Such model-based technique can also be adapted to include a resistor in series with the diode in the lumped model of FIG. 5A to handle aortic stenosis. As embodied herein, TEE or another imaging-derived technique can be used to determine ventricular unstressed volume (V0) for dilated hearts.

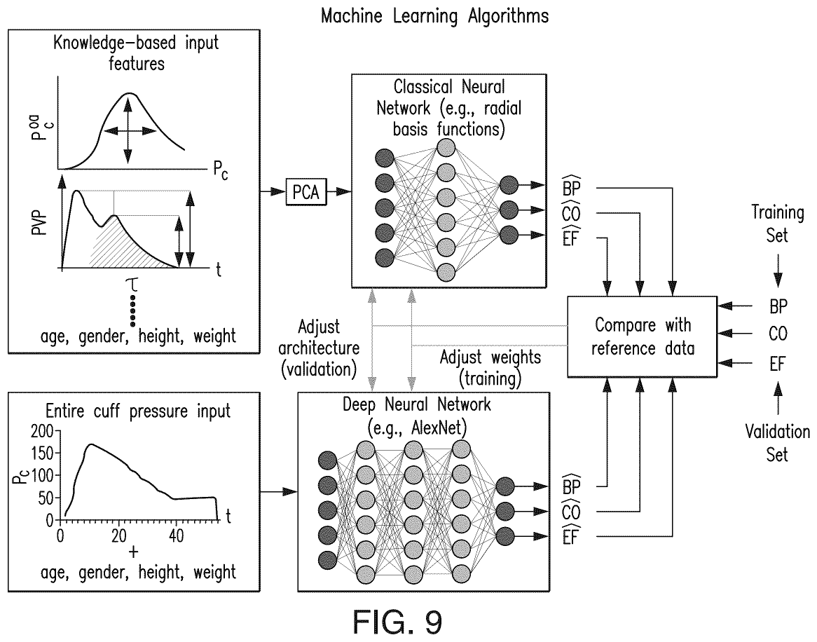

As embodied herein, the processor can be configured to include machine learning techniques, for example as shown in FIG. 9. For example, the processor can include machine learning techniques for classical learning and/or deep learning. For classical learning, candidate features can be extracted as input, for example using physiologic insight. The input can include the oscillogram width, peak location, systolic and diastolic areas of the deflation, sub-diastolic PVP waveform, BP, τ, compliance estimates via the above techniques, patient age, size, gender, or combinations thereof. As embodied herein, feature dimensionality reduction can be applied using principal components analysis as an example. For example, a stepwise regression model with the reference measurement as output can be used to determine the input features and coefficients of the linear model. Classical neural networks, such as and without limitation a multilayer perceptron or radial basis function network, can be used to capture any nonlinearity. For example, the machine learning technique can include a linear activation function in the output layer and select an activation function in a hidden layer structure by evaluating certain functions, including sigmoid and leaky rectified linear activation functions. For illustration only and not limitation, a backpropagation using the Levenberg-Marquardt technique can be used for network training. As embodied herein, the network depth can increase, and hyperparameters can be determined for example using a portion of the training data for validation or a cost function with regularization for a number of parameters, such as weight decay or minimum description length.

In certain embodiments, the techniques described herein for determining the blood pressure, cardiac output, left ventricular ejection fraction, and/or pulse pressure variation of a subject using the cuff device can further include training the cuff device using machine learning and/or deep learning. In non-limiting embodiments, the cuff device can be trained based on a training dataset. The training dataset can include, for example and without limitation, a cuff pressure dataset, a blood pressure dataset, a cardiac output dataset, or a left ventricular ejection fraction dataset collected using a medical device. In some embodiments, the medical device can include a radial artery catheter, a pulmonary artery catheter, a trans-esophageal echocardiography probe, the arm cuff device, electrodes, a mechanical ventilator, or combinations thereof.

For deep learning, for purpose of illustration and not limitation, convolutional neural networks (CNNs) with the entire cuff pressure waveform as input can be used. The CNNs can be used for deep learning as oscillograms are formed by filtering the cuff pressure waveform. As embodied herein, certain network architectures, such as AlexNet, can be used for deep learning. For example, an adapted network architecture, such as AlexNet with five convolution and two fully connected layers, can be used. The adapted network architecture can be further modified to include an activation function, such as LeakyReLU, for the entire network and batch normalization in all convolution layers to promote stable network training and avoid overfitting and reduce the size of the fully connected layer to match the number of latent network features. As embodied herein, the network architecture can have fewer convolution layers and kernels per layer with smaller kernel size. For example and without limitation, for each architecture, three CNNs can be used to estimate each parameter, or can include pre-training and a CNN with multi-task learning to estimate all variables, at least in part because the vital features per variable can overlap. Certain tools, such as and without limitation, NVIDIA Titan Xp GPU and PyTorch libraries and ADAM least-squares optimization, can be used for network training and techniques for hyperparameter determination described herein.

As embodied herein, the quality of CNNs can be assessed. For example, the latent feature space can be assessed using the t-distributed stochastic neighbor embedding technique to determine if the manifold relating the latent features to the parameters is smooth. The certain features for estimating the parameters can also be assessed using the gradient-weighted class activation mapping technique for comparison to known physiology.

Additionally, and as embodied herein, the processor can perform a technique to estimate PPV from the sub-diastolic PVP waveform. The technique can exclude arrhythmia episodes based on significant pulse interval variability and detect the respiratory period using the PVP waveform spectrum over its physiologic range. PPV can be determined using the PVP amplitudes per the standard formula, and any free parameters can be determined by measuring a greatest agreement with invasive PPV using the training data.

For illustration and not limitation, the techniques can be adjusted for balancing capabilities and convenience. For example, if the sub-diastolic PVP waveform does not offer significantly more accurate CO and EF estimates, techniques that can be performed without constant cuff pressure can be selected. As embodied herein, the microcontroller can be configured to implement the techniques in real-time and verify the code with the training data.

For purpose of illustration and confirmation of the disclosed subject matter, the performance of the exemplary cuff devices described herein can be validated for example by comparing the outputs of the exemplary cuff device with reference measurements. The reference measurements can have similar inclusion and exclusion criteria to the training data. For example, the inclusion criteria of the training data can be data of adults with radial/brachial and pulmonary artery catheters and trans-esophageal echocardiography (TEE) probe in place as part of clinical care. The exclusion criteria of the training data can be significant aortic regurgitation, mechanical cardiac support, or significant tricuspid regurgitation. As embodied herein, the reference measurements can be BP, CO, and/or EF data, which can be recorded via arterial catheters, pulmonary artery catheters, and echocardiography, including before and after clinical interventions in surgery and intensive care patients.

### EXAMPLES

### Example 1

Multi-parameter hemodynamic monitoring can be utilized for surgery and intensive care patients to detect hypotension, identify its cause, and guide fluid, pressor, and inotropic interventions (e.g., goal-directed therapy). However, measurement of blood pressure (BP), cardiac output (CO), and left ventricular ejection fraction (EF) currently requires multiple devices that are invasive, manual, or specialized. The disclosed subject matter provides methods and systems for multi-parameter hemodynamic monitoring using a wearable device configured as an automatic arm cuff device. The disclosed automatic arm cuff device was used for multi-parameter hemodynamic monitoring using various techniques.

The techniques can be developed using training data. FIG. 7 shows example patient data that can be collected for training data. For example, data of patients admitted for surgery and intensive care can be eligible as training data. The inclusion criteria can be adults with radial/brachial and pulmonary artery catheters and trans-esophageal echocardiography (TEE) probe in place as part of clinical care. The exclusion criteria can be significant aortic regurgitation (as the techniques estimate total rather than forward flow), mechanical cardiac support (which distorts arterial waveforms), or significant tricuspid regurgitation (which is a contraindication of some reference CO devices).

The patient data can be collected using other devices. For example, a custom device can be placed on the free arm, a data acquisition system (e.g., MP160, Biopac) can record all data at greater than 250 Hz sampling rate. The disclosed device can be used for collecting training data. For example, the device can perform two consecutive measurements (e.g., 1-min apart) every 15-min in the OR and every hour in the ICU. In the OR, TEE can be used to measure EF before and after interventions given as part of clinical care, including surgical landmarks (e.g., venous clamping and unclamping for a liver transplant, cardiopulmonary bypass for coronary artery bypass surgery) and therapy (e.g., fluids, pressors, inotropes). In the ICU, trans-thoracic echocardiography (TTE) can be used every four hours during the day. The patient characteristics, time-stamped annotations for the interventions, and echocardiography measurements can be documented. Data with unreliable reference measurements (e.g., artifact-contaminated BP waveforms, unsteady continuous thermodilution CO, and poor-quality echocardiography images) can be removed from the training data.

FIG. 8 shows an example of cuff pressure pre-processing. A technique to detect the beats, motion, and other artifacts, and arrhythmia in the cuff pressure waveform was developed. The technique can identify insurmountable cuff pressure waveform artifacts, the beats of the relatively clean cuff pressure waveform segments, and atrial fibrillation (AF) or another major arrhythmia with or without the aid of the ECG waveform. These techniques can provide the estimates of BP, CO, and EF, “—” in the case of artifact or misdetections, “afib” in the case of AF, or “arrhythmia” in the case of another major rhythm disturbance. The disclosed device can alternatively estimate the variables during challenging arrhythmias. For accurate construction of the oscillogram, the disclosed device can include a regression model to predict each pulse amplitude of the sub-diastolic PVP waveform via the two preceding pulse intervals and to subtract out the AF-induced variability in the oscillogram. Machine learning techniques can be used to estimate the three variables from the cuff pressure waveform as shown in FIG. 9.

An example of a patient-specific technique for brachial cuff BP estimation according to the disclosed subject matter is provided. Techniques for estimating BP from the oscillogram obtained with an automatic cuff device can employ fixed ratios. Mean BP (MP) is estimated as the cuff pressure at which the oscillogram is maximal, and systolic BP (SP) and diastolic BP (DP) are estimated as the cuff pressures at which the oscillogram is some fixed ratios of its maximum. Techniques of commercial devices can use variants of the fixed-ratio technique. Such techniques can be based on population averages, which can be suitable in typical patients. The accuracy of the devices can degrade in patients with high pulse pressure (PP=SP−DP) due to arterial stiffening, which occurs with aging.

The disclosed patient-specific technique can estimate BP from the oscillogram. The oscillogram can be represented with a physiologic model, and then the model parameters, which denote SP and DP and define arterial stiffness, can be estimated by fitting the model to the oscillogram. The method can be specific to the patient at the time of measurement in the sense that both arterial stiffness and BP are measured. Hence, accuracy can be maintained over a wide PP range. FIG. 3A shows an example patient-specific technique. The oscillogram (e.g., vertical difference between the two red envelopes) can be represented with a model accounting for the nonlinear arterial blood volume-transmural pressure relationship 301 (FIG. 3A).

The model parameters are SP and DP and define arterial stiffness [a, b, c, e]. In terms of the arterial compliance curve (i.e., derivatives of the nonlinear relationship with respect to transmural pressure), a reflects the transmural pressure at which the curve is maximum; b and c denote the width of the curve and extent of asymmetry about its maximum, and e goes with the amplitude of the curve. The parameter e is also determined by the reciprocal of the cuff compliance [k], which is assumed constant as justified by experimental data (see nearly linear cuff pressure-air volume relationships for two types of cuffs in FIG. 3A). Consistent with experimental data, a is set to near 0 mmHg, and b is constrained based on the value of c to yield a right-skewed compliance curve. Then, the remaining four patient-specific parameters are estimated by least-squares fitting of the model to the oscillogram 302 in FIG. 3A.

Validation: the technique was evaluated using data collected from 145 mainly cardiac catheterization patients. The data included arm cuff pressure waveforms via a high-end office device (e.g., WatchBP Office, Microlife, Switzerland or VP-1000, Omron Colin, Japan) and reference BP via mainly brachial artery catheterization. Reference PP ranged from normal to high in these data due to varying degrees of arterial stiffening. 57 patient records were used to optimize the technique (e.g., determine a value and b-c constraint), and the other 88 patient records were used to compare its accuracy to the office device.

FIG. 3B shows the brachial BP errors (e.g., after removing the bias component) for the patient-specific technique and office device. In subjects with normal PP, the patient-specific technique produced comparable errors to the device. However, in subjects with high PP, the patient-specific technique yielded significantly lower error magnitudes by 33% and 50-67% fewer large errors relative to the device. In sum, the patient-specific technique improved the accuracy of the automatic cuff device in patients with arterial stiffening without compromising accuracy in patients with compliant arteries.

According to another example, a physiologic technique for indirect measurement of central BP using an automatic arm cuff is provided. Brachial SP and PP are amplified relative to central (i.e., near the heart) SP and PP. As shown in FIG. 4A, this can be mainly caused by arterial wave reflection. As such, central BP reflects cardiac performance. For this reason, central BP can provide superior clinical value over brachial BP. However, central BP is far more difficult to measure directly.

The physiologic technique can measure central BP indirectly from the cuff pressure waveform obtained with a standard automatic arm cuff device. A brachial BP waveform can be constructed from the cuff pressure waveform during the deflation period and then be converted to the central BP waveform via a variable transfer function model of wave reflection. FIG. 4B overviews an example physiologic technique, which can invoke three sub-techniques. First, the patient-specific technique can be applied to estimate brachial SP and DP. Second, as shown in FIG. 4C, an ensemble averaging/calibration technique can be applied to derive a brachial BP-like waveform. A constant-amplitude cuff pressure waveform or “pulse volume plethysmography (PVP)” waveform can be extracted from the variable-amplitude cuff pressure waveform via ensemble averaging of normalized waveform beats over the lower cuff pressure regime or transmural pressure regime around 0 mmHg, wherein the arterial compliance can be relatively constant and then scaled to the brachial SP and DP. For example, the variable amplitude cuff pressure oscillation waveform can be analyzed over the cuff pressure range, which can extend from (i) the minimum cuff pressure analyzed by the patient-specific technique to (ii) the minimum cuff pressure analyzed plus 40 mmHg. The waveform beats can be detected. To eliminate anomalies, all waveform beats of lengths within about 30% of the average beat length can be selected. Alternatively, for example if fewer than three waveform beats meet this criterion, the three waveform beats with lengths closest to the average beat length can be selected. Each selected waveform beat, including about 250 msec intervals before the first foot and after the last foot, can be equalized by normalization to peak amplitude of one and feet amplitudes of zero. In non-limiting embodiments, time normalization can also be employed, if necessary, to further equalize the waveform beats. To further eliminate anomalies, a template waveform beat can be constructed by computing the ensemble median of all selected waveform beats over the minimum beat length and then applying the same normalization. The three waveform beats with root-mean-squared-error (RMSE) less than about 0.5 relative to the template waveform beat that can be nearest to the minimum cuff pressure can be selected. Alternatively, for example if less than three waveform beats meet this criterion, the three waveform beats with the lowest RMSEs can be selected. The ensemble average of the selected waveform beats can be computed over the minimum beat length and can be normalized to yield the deflation PVP waveform. This waveform can then be scaled to brachial SP and DP to yield a brachial BP-like waveform. Third, as is shown in FIG. 4D, a variable transfer function technique can be used to convert the brachial BP-like waveform Pb(t) to the central BP waveform Pc(t). The transfer function can be defined in terms of a tube-load model of wave reflection with parameters denoting pulse transit time Td and the wave reflection coefficient F (see for example the transfer function in time-domain in FIG. 4D). The F parameter is set to a nominal value, as the transfer function is often insensitive to it, while Td, which can impact the extent to which the transfer function reduces BP amplification, is predicted based on its well-known inverse relationship with Mean BP (MP) (see FIG. 4D). As such, MP using the average of the brachial BP-like waveform can be used to predict Td. The fully defined transfer function can then be applied to this waveform to derive the central BP waveform.

Validation: the technique was assessed using data collected from 87 cardiac catheterization patients. The data included cuff pressure waveforms via the office device described herein and reference central BP via aortic catheterization. 36 patient records were used to optimize the technique (e.g., determine the F value and Td prediction equation) and the other 51 patient records to test its accuracy. FIG. 4E shows the central BP measurements of the physiologic technique versus the reference values. The technique yielded central BP bias and precision errors [μ and σ] of <3 mmHg in magnitude and <9 mmHg, which nearly satisfies the regulatory limits of 5 and 8 mmHg. In sum, the physiologic technique can allow central BP to be measured reliably and in the same way as traditional brachial cuff BP. While non-invasive devices for central BP monitoring exist, none offer such convenience.

According to another example, a multi-beat analysis technique for CO estimation from a radial BP waveform is provided. “Un-calibrated” pulse contour devices can be used for estimating CO from only a radial BP waveform. As shown in FIG. 5A, at a high level, these devices extract PP or the arterial “RC” time constant τ by fitting an exponential to the BP diastolic decay to estimate CO to within an arterial compliance C scale factor. However, wave reflection can cause peripheral PP to change irrespective of CO due at least in part to vasoconstriction/vasodilation and also obscure exponential diastolic decays in peripheral BP waveforms, which can make accuracy a challenge.

The multi-beat analysis technique can estimate CO from the radial BP waveform. The disclosed device can model (rather than ignore) confounding wave reflection. As shown in FIG. 5B, the BP response to a single heartbeat can be estimated from a waveform segment over multiple beats. Then, τ is determined by fitting an exponential to the tail end of this response once the faster wave reflection vanishes. Next, C can be determined from BP and patient age, height, weight, and gender using a proprietary formula. Then, CO can be computed via Ohm's law.

For example, the single heartbeat BP response can be estimated in two steps. First, a waveform indicating the heartbeats x(t) can be formed as an impulse train in which each impulse is located at the minimum or foot of the BP waveform y(t) and scaled by the ensuing PP (FIG. 5B). Then, an impulse response h(t) can be identified, which when convolved with x(t) fits y(t) via autoregressive exogenous input least-squares identification. By definition, h(t) represents the BP response to a single heartbeat.

For purpose of illustration only and confirmation of the disclosed subject matter, the multi-beat analysis technique was verified in animals with reference aortic flow probes, healthy humans subjected to central hypovolemia, and critically ill patients with reference pulmonary artery catheters. The technique was incorporated into a commercial device and provided greater accuracy in estimating CO changes in patients.

An example model-based technique for EF estimation from a BP waveform is provided. The model-based technique can estimate EF from a BP waveform. The disclosed device can model both the left ventricle and arteries. The model can then be fitted to the entire BP waveform to estimate the model parameters to within C scale factors. The C term can cancel out in the ensuing computation of EF to yield an absolute measurement in units of percent. FIG. 6A-6C show an example model-based technique. The technique was designed for application to the central BP waveform, which, unlike peripheral BP waveforms, often exhibit exponential diastolic decays. This BP waveform Pc(t) is represented with a lumped model of the left ventricle and arteries (see FIG. 6A). The ventricle is characterized by the well-known variable elastance (VE, reciprocal of compliance) model in which elastance varies over time E(t) to drive blood flow and unstressed volume V0 is non-zero; the aortic valve is modeled with an ideal diode (as valve disease is assumed absent), and the arteries are embodied by the popular RC circuit (“Windkessel”) model. E(t) is parameterized via a raised cosine (see FIG. 6B). The model has four parameters: τ=RC, CEₘₐₓ, Ts, CE(t_{be}), where Eₘₐₓ is the maximum VE over a cardiac cycle; Ts is the time duration to reach Eₘₐₓ from the minimum VE, and t_{be} is the start time of the ejection interval (see FIG. 6B). The parameters are estimated from Pc(t) in two steps. First, τ is determined by least-squares fitting of an exponential to its diastolic interval (see FIG. 6B). Then, CEₘₐₓ, Ts, and CE(t_{be}) are estimated from the ejection interval of Pc(t) and τ by least squares matching of both sides of the governing model 601 in FIG. 6C.

EF is computed from the CE(t) estimate and Pc(t) by deriving stroke volume (SV) and ventricular end-diastolic volume (EDV) to within C scale factors based on the model equations and then taking their ratio to nullify the common C term 602 in FIG. 6C.

V0 can be set to a small value (e.g., 15 mmHg) or measured via echocardiography EF for patients with dilated hearts. Only one such imaging measurement is needed over a monitoring period, as V0 can change slowly over time with ventricular remodeling.

For purpose of illustration only and confirmation of the disclosed subject matter, the model-based technique was evaluated in six animals with intermittent reference echocardiography measurements during various hemodynamic interventions. As shown in FIG. 6D, the technique achieved an absolute EF error of only 5.6%. In sum, the model-based technique can allow—for the first time—continuous EF monitoring via only an arterial waveform. EF can also be tracked from an invasive radial BP or non-invasive blood volume waveform, thereby indicating reproducibility of the idea.

The disclosed device can be further validated using reliable reference measurements in patients. The device can be tested with new surgery and intensive care patients using the same inclusion/exclusion criteria. The root-mean-squared-error (RMSE=sqrt(j2+a2) can be used, where j and a are the bias and precision errors) of a test device against a reference device and concordance rate between these devices (percentage of changes in two measurements of the same sign). The null hypotheses of the test can be: (i) new device BP RMSE>existing arm cuff device BP RMSE; (ii) new device CO RMSE normalized by reference CO>volume-clamp device CO RMSE normalized by reference CO+10%; (iii) new device CO concordance rate+5%<volume-clamp device CO concordance rate; and (iv) new device EF RMSE normalized by reference EF>10%. The 5-10% margins are based on the known error in the reference devices and clinically meaningful differences (e.g., 10-15% CO changes are not actionable). A null hypothesis at a one-sided 0.025 level of significance with a correction can be rejected for multiple comparisons. The patient sample size can be determined by creating thousands of bootstrapped samples using the training data results under a normality assumption (e.g., bias and precision errors of the new techniques and competing devices, correlation of the errors within a patient and between these techniques and devices, and mean and standard deviation of the reference values). The number of patients that can yield a detection probability of >0.8 for all of the tests can be selected.

Data from each patient can be collected as described above with the new device outputting values. The data with unreliable reference measurements can be discarded, and the cuff pressure waveforms can be screened for major artifacts and arrhythmias. The accuracy metrics of the new and competing devices can be computed. A random-effects model can be used with a compound symmetry assumption for the within-patient covariance matrix to account for the repeated measures per patient in calculating the RMSEs and a 15% exclusion zone in computing the CO concordance rates. The metrics can be statistically compared using cluster bootstrapping to preserve the correlated data structure. The sensitivity and specificity of the device in detecting major artifacts and arrhythmia can be computed. The device can reject at least one null hypothesis, and the accuracy in artifact and arrhythmia detection can be >0.9.

### Example 2

One example of a methodology employed by automatic cuff blood pressure (BP) measurement devices is oscillometry. These devices vary the external pressure of the artery via cuff inflation/deflation while measuring the cuff pressure. Systolic and diastolic BP (Ps and Pd) are estimated from the oscillogram, e.g., the function relating the amplitude of the cuff pressure oscillations (ΔO, which indicate blood volume pulsations) to the applied cuff pressure (Pc). Empirical methods are used to estimate the BP, which may be accurate only for a limited BP range. A mathematical model of the oscillogram can improve the BP estimation. A parametric model may be fitted to the oscillogram to estimate BP in a principled and general way. An effective model of the oscillogram is as follows:

ΔO=kf(Pₛ−P_{c})−kf(P_{d}−P_{C}) (1)

where f(⋅) is the arterial blood volume-transmural pressure relationship and k is a scale factor to map blood volume oscillations to cuff pressure oscillations. The derivative of f(⋅) with respect to P_{c}(g(⋅)) represents the brachial artery compliance curve. This curve can be parametrized to fit the model to the oscillogram. However, the best parametric function is unknown. Here, eight parametric functions were compared for representing the brachial artery compliance curve.

In this example, de-identified data collected from a total of 147 human subjects at the Taipei Veterans General Hospital (Taiwan) were used. Of those, 128 were adult patients admitted for cardiac catheterization. Briefly, all patients had normal sinus rhythm and inter arm BP cuff differences of no more than 3 mmHg. Both invasive BP and cuff pressure waveforms were simultaneously recorded at 250 Hz during baseline and/or sublingual nitroglycerin administration. The other 19 subjects were healthy adults who had two pairs of cuff pressure waveforms and auscultation measurements recorded. The cuff pressure waveforms were visually screened, and those with substantial artifact or incomplete oscillograms were excluded. A total of 215 cuff pressure waveforms remained for analysis. The oscillograms were first extracted from the cuff pressure waveforms. The model was then optimally fitted to the oscillograms in the least-squares sense using eight different parametric functions for g(⋅). The functions were unimodal and right-skewed curves as justified by experimental data. The measured systolic BP and diastolic BP were input for Ps and Pd in the model. Four unknown parameters remained representing the peak position of the brachial artery compliance curve, the left and right curve widths with respect to the peak position, and the curve amplitude. The model fits were evaluated in terms of the root-mean-square (RMS) of the fitting error normalized by the RMS of the measured oscillogram (NRMSE). Per initial analyses, the peak compliance location did not have any significant influence on the fitting and hence was set to 0 mmHg in line with experimental data. Empirical evaluation of the models helped define the physiologically appropriate ranges for the three remaining parameters. A one-way repeated measures ANOVA was performed to determine any significant differences between the model fits.

For purpose of illustration and confirmation of the disclosed subject matter, the Weibull and exponential-linear parametric functions were able to best fit the oscillograms with respective NRMSEs of 8.2±0.3% (mean±SE) and 8.7±0.3%. These exemplary functions are as follows:

$$g ( P ) = \frac{dc}{b} {( \frac{P - a}{b} )}^{c - 1} exp [ - {( \frac{P - a}{b} + {( \frac{c - 1}{c} )}^{\frac{1}{c}} )}^{c} ] \qquad ( 2 )$$

$$g ( P ) = d e \frac{P - a}{b} u ( - P + a ) + d e \frac{- ( P - a )}{c} u ( P - a ) \qquad ( 3 )$$

The one-way repeated measures ANOVA showed that the model fits were different overall (p<0.05). The above-mentioned functions were observed to predict the data significantly better than three of the other functions (Burr3, Burr12 & Fisk) and the Drzewiecki model. The remaining two functions were Gaussian and exponential functions, and they were observed to produce similar fits to Eqs. (2) and (3).

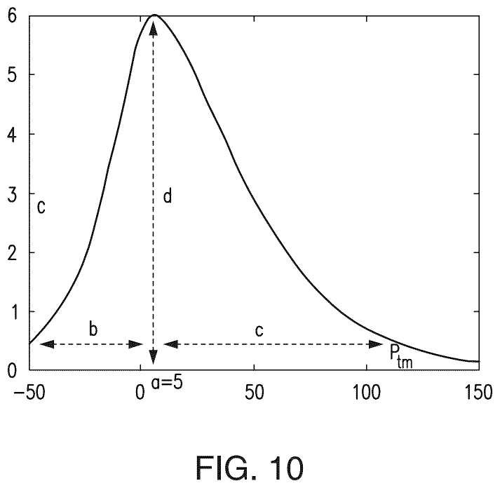

In this manner, the Weibull and exponential-linear functions of Eqs. (2) and (3) produced the best oscillogram fits of the eight functions studied. The Weibull function performed marginally better than the exponential-linear function. However, this function has a complex relationship between its model parameters and the key compliance curve characteristics. The exponential-linear function (FIG. 10), on the contrary, fits the experimental data almost as well and its parameters (a, b, c and d) correspond directly to the peak location, left and right curve widths, and peak amplitude of the compliance curve. The expo-linear model in FIG. 10 is with the peak location (a) at 5 mmHg, where curve widths are defined (b)=13 mmHg; (c)=26 mmHg and (d)=6 mmHg as peak amplitude.

Further, in contrast to the Gaussian and exponential functions, the exponential-linear function leads to simple closed-form expressions. Hence, all-in-all, the exponential-linear function may be more suitable for representing the brachial artery compliance curve.

In addition to the specific embodiments claimed below, the disclosed subject matter is also directed to other embodiments having any other possible combination of the dependent features claimed below and those disclosed above. As such, the particular features presented in the dependent claims and disclosed above can be combined with each other in other manners within the scope of the disclosed subject matter such that the disclosed subject matter should be recognized as also specifically directed to other embodiments having any other possible combinations. Thus, the foregoing description of specific embodiments of the disclosed subject matter has been presented for purposes of illustration and description. It is not intended to be exhaustive or to limit the disclosed subject matter to those embodiments disclosed.

It will be apparent to those skilled in the art that various modifications and variations can be made in the method and system of the disclosed subject matter without departing from the spirit or scope of the disclosed subject matter. Thus, it is intended that the disclosed subject matter include modifications and variations that are within the scope of the appended claims and their equivalents.

What is claimed is:

1. A method for determining a cardiac output of a patient using a cuff device comprising:
measuring a cuff pressure waveform of a subject during at least one of inflation and deflation of the cuff device;
computing systolic and diastolic blood pressure from the cuff pressure waveform using the cuff device;
constructing a blood pressure waveform from the systolic and diastolic blood pressure and cuff pressure waveform using the cuff device;
computing brachial artery compliance from the cuff pressure waveform; and
computing the cardiac output from at least one of the blood pressure waveform and brachial artery compliance using the cuff device.

2. The method of claim 1, wherein the cuff pressure waveform is measured during slow cuff inflation and/or deflation of the cuff device followed by maintaining a cuff pressure at a sub-diastolic level, wherein a rate of the slow cuff inflation and/or deflation is from about 2 to about 3 mmHg/second, and wherein the cuff pressure at the sub-diastolic level is from about 40 to about 60 mmHg for about 10 to about 30 seconds.

3. The method of claim 1, wherein the cardiac output is computed based on additional information of the subject, wherein the additional information is selected from the group consisting of age, height, weight, gender of the subject, or combinations thereof.

4. The method of claim 1, wherein the brachial artery compliance is determined based on a ratio of peak-to-peak amplitude of the sub-diastolic pulse volume plethysmography (PVP) waveform to a pulse pressure or a ratio of peak-to-peak amplitude of the PVP waveform at a fixed transmural pressure to the pulse pressure.

5. The method of claim 1, wherein the additional information, blood pressure values, and/or formula to compute arterial compliance factor from the brachial artery compliance is defined through training data.

6. The method of claim 1, wherein a common feature value is estimated from the constructed blood pressure waveform, and wherein the cardiac output is computed based on the common feature value.

7. A method for determining a cardiac output of a subject using a cuff device comprising:
measuring the cuff pressure waveform during cuff inflation and deflation of the cuff device;
computing an ensemble-averaged beat of a blood pressure waveform from the cuff pressure waveform; and
computing the cardiac output from the ensemble-averaged blood pressure waveform beat.

8. The method of claim 7, further comprising computing brachial artery compliance from the cuff pressure waveform using the cuff device.

9. The method of claim 8, wherein the cardiac output is computed based on additional information of the subject, wherein the additional information is selected from the group consisting of the brachial artery compliance, age, height, weight, gender of the subject, or combinations thereof.

10. The method of claim 7, wherein the ensemble-averaged beat is formed from beats in a low cuff pressure range or from beats around or near a zero transmural pressure range.

11. The method of claim 8, wherein the brachial artery compliance is determined based on a ratio of peak-to-peak amplitude of a pulse volume plethysmography (PVP) waveform at a fixed transmural pressure to pulse pressure.

12. The method of claim 9, wherein a formula to compute the cardiac output from the brachial artery compliance, blood pressure values, blood pressure waveform beat features, and/or the additional information is defined through training data.

13. The method of claim 7, wherein the blood pressure waveform is constructed without maintaining a cuff pressure at a sub-diastolic level.

14. A method for determining left ventricular ejection fraction of a subject using a cuff device comprising:
measuring a cuff pressure waveform during cuff inflation and deflation of the cuff device;
computing a systolic and diastolic blood pressure from the cuff pressure waveform using the cuff device;
constructing a blood pressure waveform from the systolic and diastolic blood pressure and the cuff pressure waveform; and
computing the left ventricular ejection fraction from systolic and diastolic intervals of the constructed blood pressure waveform.

15. The method of claim 14, further comprising imaging the left ventricular ejection fraction for computing a subsequent left ventricular ejection fraction in the subject, wherein the subject has a dilated heart.

16. The method of claim 14, wherein the left ventricular ejection fraction is determined from the constructed blood pressure waveform using classical or deep machine learning.

17. The method of claim 14, wherein the left ventricular ejection fraction is determined from a constructed blood pressure waveform using a physiologic model representing a left ventricle and arterial system.

18. The method of claim 17, wherein the left ventricle system is characterized by a variable elastance model.

19. A method for determining pulse pressure variation of a subject using a cuff device comprising:
measuring a cuff pressure waveform of the subject while maintaining a cuff pressure at a sub-diastolic level;
detecting a peak-to-peak amplitude of each beat of the cuff pressure waveform;
computing a respiratory period of the cuff pressure waveform; and
computing the pulse pressure variation from the peak-to-peak amplitudes and respiratory period.

20. A method for determining a systolic and diastolic blood pressure using a cuff device comprising:
measuring a cuff pressure waveform of a subject during at least one of inflation and deflation of the cuff device;
constructing an oscillogram as a function relating variable cuff pressure oscillation amplitudes to cuff pressure;
representing the oscillogram with a parametric model with parameters comprising the systolic and diastolic blood pressure and defining a brachial artery compliance curve, wherein the brachial artery compliance curve is further represented with an exponential-linear parametric function;
fitting the parametric model to the oscillogram to estimate the model parameters; and
determining the systolic and diastolic blood pressure and the brachial artery compliance curve using the model parameter values.
